(19) United States
(12) Patent Application Publication
(10) Pub. No.: US 2011/0152715 A1
(43) Pub. Date: Jun. 23, 2011

(54) BIOPSY NEEDLE WITH VACUUM ASSIST

(75) Inventors: Dennis J. Delap, Bloomington, IN (US); Cleve S. Koehler, Ellettsville, IN (US); Robert S. Childress, Solsberry, IN (US); Randy Joe Myers, Bloomington, IN (US)

(73) Assignee: Cook Incorporated, Bloomington, IN (US)

(21) Appl. No.: 12/645,106

(22) Filed: Dec. 22, 2009

(51) Int. Cl. A61B 10/02

(52) U.S. Cl. 600/566

(57) ABSTRACT

A surgical cutting device comprises a cannula attached to an actuation mechanism that moves the cannula from a cocked position to a cutting position, a stylet including a distal portion having a sharp distal end, a sample collection region, a vacuum port, and a lumen extending from the vacuum port to the sample collection region such that the sample collection region is in fluid communication with the first vacuum port. When the cannula is in the cocked position, the sample collection region of the stylet is disposed distal of the cannula. The device also includes a fixed volume vacuum source. When cannula is in the cocked position, the vacuum port is not in fluid communication with the fixed volume vacuum source, and when said cannula is in the cutting position, the vacuum port is in fluid communication with the fixed volume vacuum source.

### BACKGROUND

The present invention relates generally to medical devices and more particularly to a surgical device for biopsy sampling of tissue.

Biopsy is the removal and study of body tissue for medical diagnosis. Typically, physicians obtain biopsy samples in order to detect abnormalities, such as cancer, and to determine the extent to which cancerous tissue has spread. Generally, tissue samples are acquired from different areas of the body using biopsy instruments. Common biopsy instruments comprise a two-part needle assembly, commonly referred to as a stylet and cannula, operated by a spring-loaded handle of the type disclosed in U.S. Pat. No. 5,538,010, the entirety of which is incorporated herein by reference. In use, the biopsy device is inserted through the skin to the target biopsy site with the cannula in the “cocked,” or retracted position, and the stylet in the retracted position. The stylet is then advanced out of the cannula to its deployed position, which is distal of the distal end of the cannula. In this deployed state, the target tissue is exposed to the sample collection region of the stylet, thereby allowing the tissue to prolapse into the sample collection region. The cannula is then “fired” by releasing a triggering mechanism, which causes the cannula to spring forward toward the distal end of the sample collection region, thereby severing any tissue present in the sample collection region from the surrounding tissue mass. Typically, the cannula and stylet are attached to a spring-loaded handle that advances the cannula over the stylet very quickly in order to prevent the prolapsed tissue in the sample containing region from being displaced as the cannula advances over the stylet. Once the cannula is advanced, the sample is trapped between the cannula and the sample containing region of the stylet. The biopsy needle may then be withdrawn and the tissue sample recovered from the stylet.

While this system works well for a variety of biopsy procedures, the proliferation of tissue based medical diagnostic tests has increased the demand for larger and larger tissue samples. However, because biopsy is an invasive procedure that involves insertion through the skin and the removal of tissue, the larger the needle used to procure the tissue sample, the greater the discomfort to the patient and longer the healing/recovery time.

### SUMMARY

Surgical cutting devices are described which may facilitate procurement of tissue samples. The embodiments may include any of the following aspects in various combinations and may also include any other aspect described below in the written description or in the attached drawings.

In one aspect, a surgical cutting instrument may include a cannula having a distal end shaped to cut tissue. The cannula is attached to an actuation mechanism that moves the cannula from a cocked position to a cutting position. Also included is a stylet having a distal portion with a sharp distal end, a sample collection region spaced proximally away from the sharp distal end, a first vacuum port, and a lumen extending from the first vacuum port to the sample collection region such that the sample collection region is in fluid communication with the first vacuum port. The stylet is movable between a retracted position, in which an entirety of the sample collection region is disposed within the cannula in the cocked position, and a deployed position, in which at least a portion of the sample collection region is disposed distal of the distal end of the cannula in the cocked position. The surgical cutting device also includes a fixed volume vacuum source and a control handle. The control handle includes a second vacuum port. The fixed volume vacuum source is releasably attached to the control handle such that the control handle is in fluid communication with the second vacuum source.

When the stylet is in the retracted position, the first and second vacuum ports are not in fluid communication with each other, thereby sealing the fixed volume vacuum source from the lumen of the stylet. When the stylet is in the deployed position, the first and second vacuum ports are in fluid communication thereby allowing fluid flow from the sample collection region of the stylet to the fixed volume vacuum source through the lumen.

In another aspect, the movement of the stylet from the retracted position to the deployed position causes the first and second vacuum ports to come into in fluid communication with each other.

In one embodiment, the proximal portion of the stylet has a first cross sectional area and the sample containing region has a second cross sectional area, the second cross sectional area being smaller than the first. The sample containing region includes a plurality of apertures disposed an outer surface thereof, the plurality of apertures being in fluid communication with the lumen of the stylet.

In another embodiment, the sample containing region has a first surface and a second surface that are displaced from each other to form a lumen therebetween. The first surface is substantially contiguous with an outer surface of the stylet and the second surface is disposed radially inward of the outer surface of the stylet, thereby creating a space to receive a sample. The second surface includes a plurality of apertures along its length, the apertures being in fluid communication with the lumen of the stylet.

In yet another embodiment, the surgical cutting device may include a stylet comprising a distal portion having a sharp distal end, a sample collection region, a first vacuum port, and a lumen extending from the first vacuum port to the sample collection region such that the sample collection region is in fluid communication with the first vacuum port. The stylet may be disposed within the cannula. When the cannula is in the cocked position, an entirety of the sample collection region is disposed distal of the distal end of the cannula. The first vacuum port is disposed distal of a proximal end of the cannula when the stylet is in the deployed position. The surgical cutting device also includes a control handle having a second vacuum port and a fixed volume vacuum source that is releasably attached to the control handle and in fluid communication with the second vacuum port.

When the cannula is in the cocked position, the second vacuum port is sealed from the first vacuum port, thereby preventing fluid communication between the fixed volume vacuum source and the lumen of the stylet. When the cannula is in the cutting position, the first and second vacuum ports become at least partially unsealed from one another, thereby permitting fluid flow from the sample collection region of the stylet to the fixed volume vacuum source through the lumen and the first and second vacuum ports.

In one aspect, the first vacuum port is disposed distal of a proximal end of the cannula when the stylet is in the deployed position, and the control handle further comprises first and second seals. The first seal may be disposed proximal of the second vacuum port, and the second seal may be disposed distal of the second vacuum port. When the cannula is in the cocked position and the stylet is in the deployed position, the first and second seals sealingly engage a proximal portion of the cannula and sealingly engage a portion of the stylet that includes the first vacuum port. The first vacuum port may be sealed from the second vacuum port by the proximal portion of the cannula, thereby preventing fluid communication between the fixed volume vacuum source and the lumen of the stylet. When the cannula is in the cutting position, the proximal portion of the cannula disengages from at least one of the first and second seals, thereby permitting fluid flow from the sample collection region of the stylet to the fixed volume vacuum source through the lumen and the first and second vacuum ports.

In another aspect, the movement of the cannula from the cocked to the cutting position may cause the first and second vacuum ports to come into in fluid communication with each other.

In yet another aspect, the surgical cutting instrument may also include a sealing sleeve attached to a second actuation mechanism that moves the sealing sleeve from a sealed position to a released position. When the sealing sleeve is in the sealed position, the first vacuum port is sealed from the second vacuum port by the sealing sleeve, the sealing sleeve thereby preventing fluid communication between the fixed volume vacuum source and the lumen of the stylet. When the sealing sleeve is in the released position, the first and second vacuum ports become at least partially unsealed from one another, thereby permitting fluid flow from the sample collection region of the stylet to the fixed volume vacuum source through the lumen and the first and second vacuum ports. The first vacuum port is disposed within the sealing sleeve when the cannula is in the cocked position. In another aspect, the control handle may also include first and second seals, the first seal being disposed proximal of the second vacuum port and the second seal being disposed distal of the second vacuum port, respectively, wherein, when the sealing sleeve is in the sealed position, the first and second seals sealingly engage the sealing sleeve and sealingly engage a portion of the stylet comprising the first vacuum port. When the sealing sleeve is in the released position, the sealing sleeve may disengage from at least one of the first and second seals, thereby permitting fluid flow from the sample collection region of the stylet to the fixed volume vacuum source through the lumen and the first and second vacuum ports. In one embodiment, the first and second actuation mechanisms may be a single mechanism.

In one aspect, the surgical cutting device may include a stylet comprising a distal portion having a sharp distal end, a sample collection region spaced proximally away from the sharp distal end, a vacuum port disposed at a proximal end of the stylet, and a lumen extending from the vacuum port to the sample collection region such that the sample collection region is in fluid communication with the vacuum port. The stylet may be disposed within the cannula. An entirety of the sample collection region is disposed distal of the distal end of the cannula in the cocked position. The stylet is movable between a retracted position, in which an entirety of the sample collection region is disposed within the cannula in the cocked position, and a deployed position, in which the entirety of the sample collection region is disposed distal of the distal end of the cannula in the cocked position. The surgical cutting device also includes a releasably attachable fixed volume vacuum source. The vacuum port is not sealed from the lumen of the stylet. The proximal end of the stylet is shaped to attach directly to the fixed volume vacuum source. The direct attachment of the stylet and the fixed volume vacuum source may allow immediate fluid communication with the vacuum port, thereby allowing fluid flow from the sample collection region of the stylet to the fixed volume vacuum source through the stylet lumen.

In one aspect, the fixed volume vacuum source may be a sealed vacuum vial and the proximal end of the stylet has a sharp edge that pierces the sealed vacuum vial, thereby directly attaching the sealed vacuum vial to the stylet and creating fluid communication between the sealed vacuum vial and the vacuum port.

The foregoing paragraphs have been provided by way of general introduction, and are not intended to limit the scope of the following claims. The embodiments described herein will be best understood by reference to the following detailed description taken in conjunction with the accompanying drawings.

### BRIEF DESCRIPTION OF THE DRAWINGS

The embodiments may be more fully understood by reading the following description in conjunction with the drawings, in which:

FIG. 1 is an orthogonal view of a surgical cutting device according to an embodiment;

FIG. 2 is an orthogonal view of a second embodiment of a surgical cutting device;

FIG. 3 is an orthogonal view of a third embodiment of a surgical cutting device;

FIG. 4(a) is a close-up orthogonal view of a distal end portion of the surgical cutting devices of FIGS. 1-3;

FIG. 4(b) is a side elevation view of the distal end portion of FIG. 4(a);

FIG. 4(c) is a side elevation view of another embodiment of the distal end portion of FIGS. 4(a) and (b);

FIG. 5(a) is a close-up orthogonal view of another embodiment of a distal end portion of the surgical cutting devices of FIGS. 1-3;

FIG. 5(b) is a side elevation view of the distal end portion of FIG. 5(a);

FIG. 6(a) is a close-up orthogonal view of another embodiment of a distal end portion of the surgical cutting devices of FIGS. 1-3;

FIG. 6(b) is a side elevation view of the distal end portion of FIG. 6(a);

FIG. 6(c) is a cross-sectional end view at the plane X of FIG. 6(a);

FIG. 7(a) is a partial cross-sectional view of an embodiment of the surgical cutting devices of FIGS. 1-3 in which the stylet is in a retracted position and the cannula is in a cocked position;

FIG. 7(b) is a partial cross-sectional view of the embodiment of FIG. 7(a), in which the stylet is in a partially deployed position and the cannula is in the cocked position;

FIG. 7(c) is a partial cross-sectional view of the embodiment of FIG. 7(a) in which the stylet is in a fully deployed position and the cannula is in the cocked position;

FIG. 7(d) is a partial cross-sectional view of the embodiment of FIG. 7(a) in which the stylet is in the fully deployed position and the cannula is in the cutting position;

FIG. 8(a) is a partial cross-sectional view of an embodiment of the surgical cutting devices of FIGS. 1-3 in which the stylet is in the deployed position and the cannula is in a cocked position;

FIG. 8(b) is a partial cross-sectional view of the embodiment of FIG. 8(a), in which the stylet is in the deployed position and the cannula is in the cutting position;

FIG. 9(a) is a partial cross-sectional view of an embodiment of the surgical cutting devices of FIGS. 1-3 in which the stylet is in the retracted position and the cannula is in a cocked position;

FIG. 9(b) is a partial cross-sectional view of the embodiment of FIG. 9(a), in which the stylet is in the deployed position and the cannula is in the cocked position;

FIG. 9(c) is a partial cross-sectional view of the embodiment of FIG. 9(a) in which the stylet is in the deployed position and the cannula is in the cutting position;

FIG. 10(a) is a partial cross-sectional view of the embodiment of the surgical cutting devices of FIGS. 1-3 in which the stylet is in the deployed position and the cannula is in the cocked position; and

FIG. 10(b) is a partial cross-sectional view of the embodiment of FIG. 10(a) in which the stylet is in the deployed position and the cannula is in the cutting position.

### DETAILED DESCRIPTION

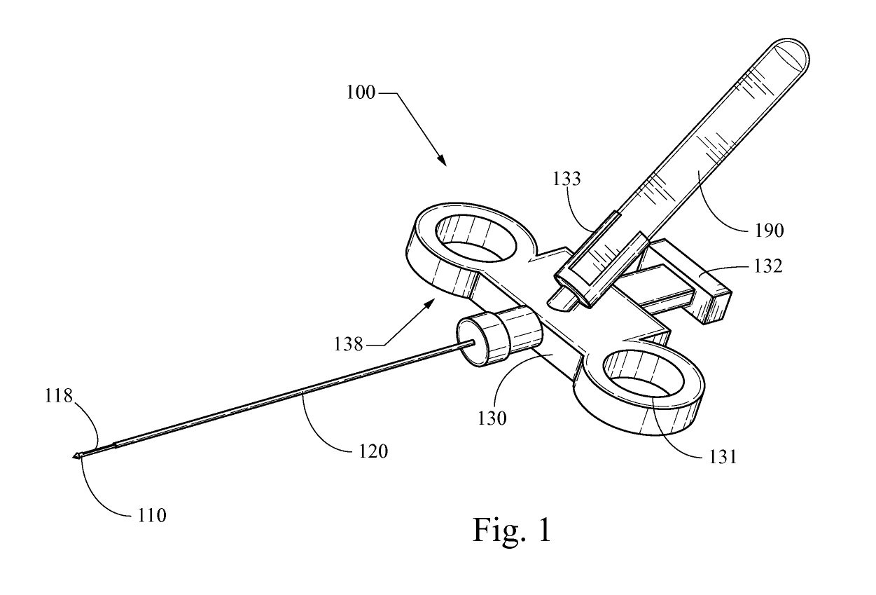

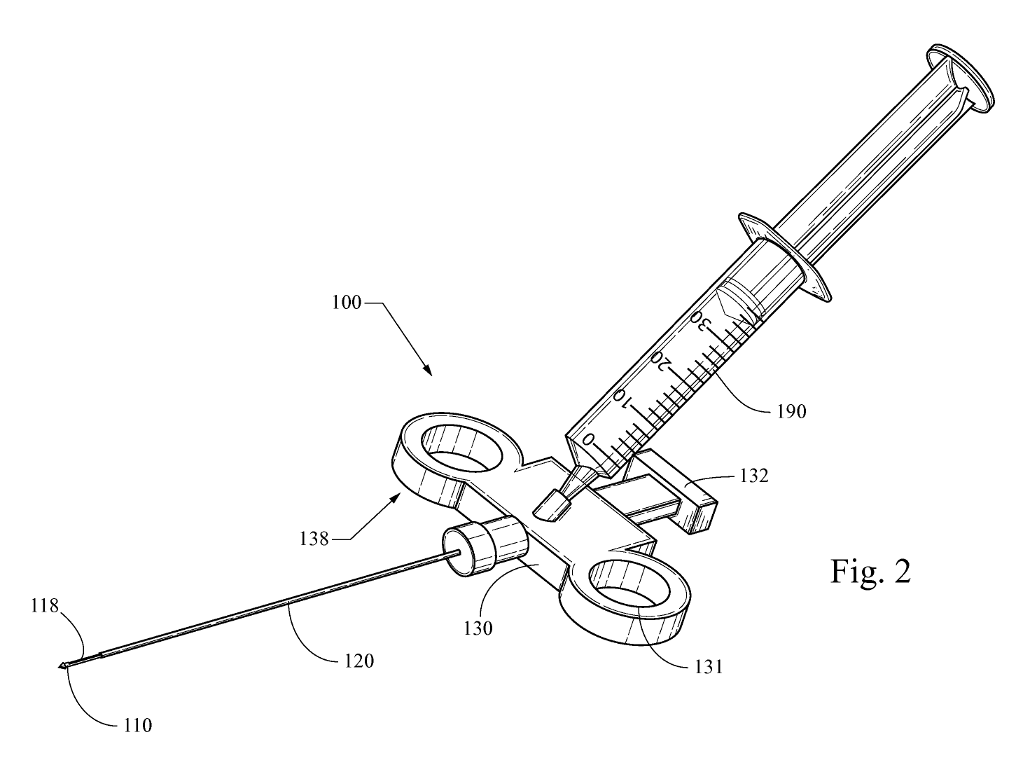

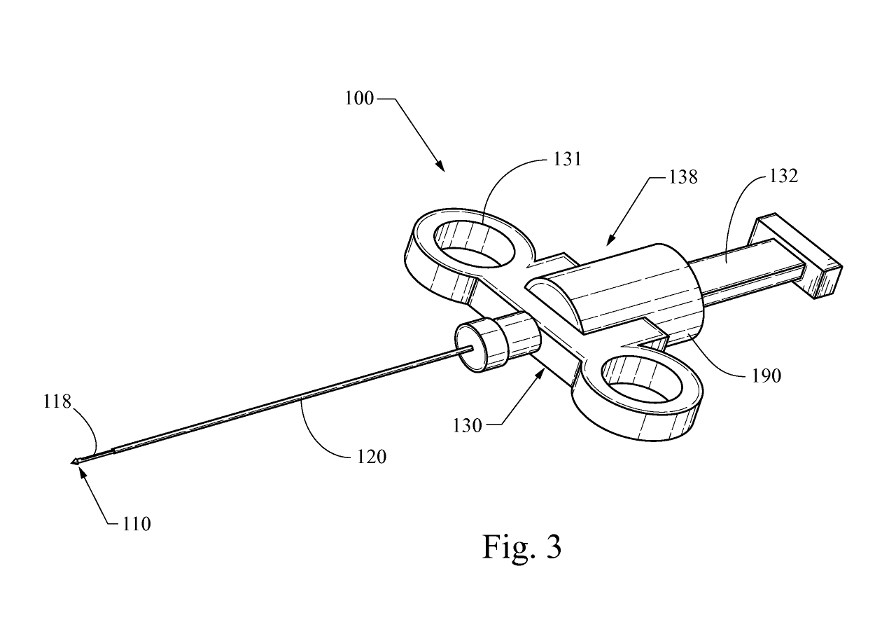

Referring now to the figures, FIGS. 1-3 illustrate embodiments of a surgical cutting instrument for biopsy sampling of tissue. More specifically, FIGS. 1 and 2 illustrate embodiments of a surgical cutting instrument 100 having a releasably attachable and replaceable fixed volume vacuum source 190, while FIG. 3 illustrates an embodiment having an integrally formed fixed volume vacuum source 190. Note that throughout this specification, like reference numbers refer to like elements in the Figures.

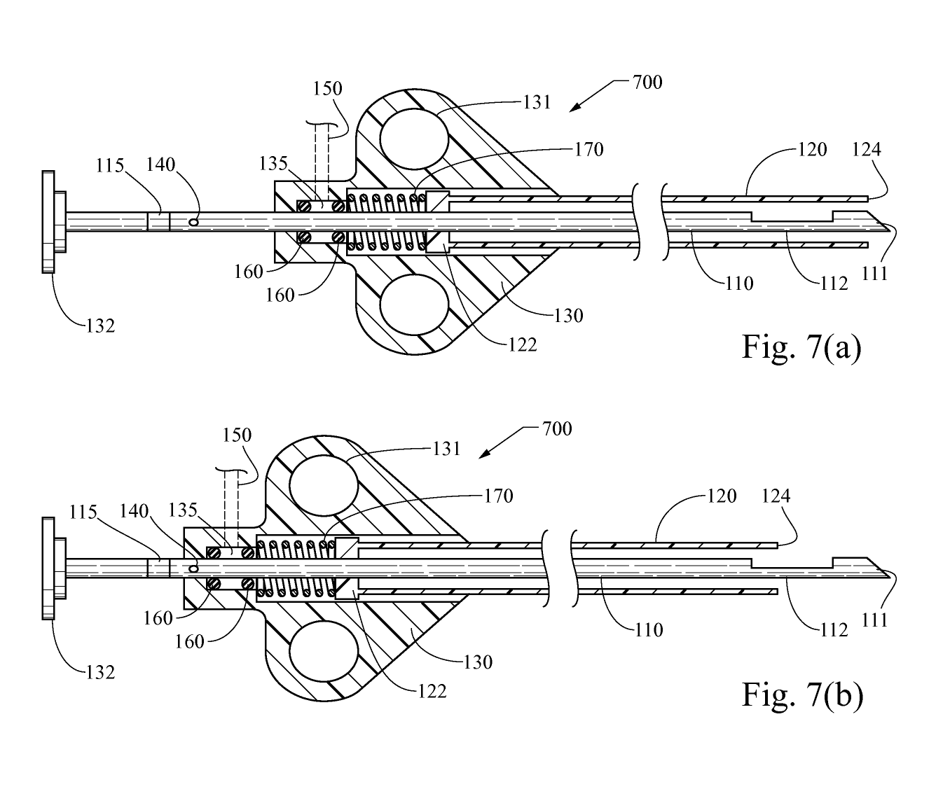

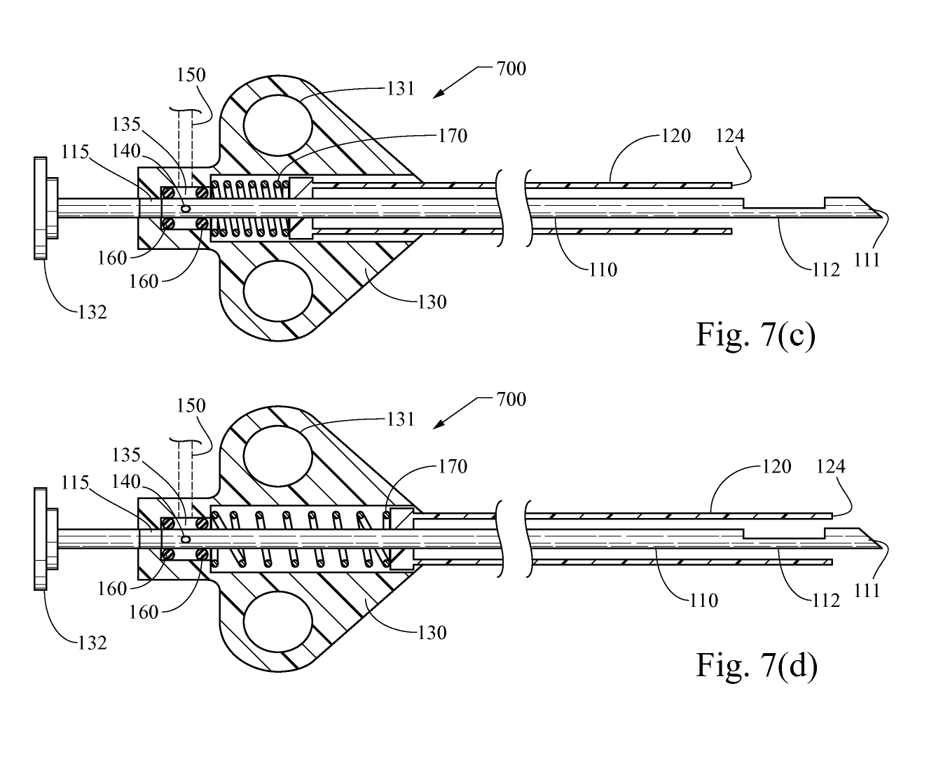

As shown in the embodiments of FIGS. 1 and 2, the surgical cutting device includes a control handle 130 connected to an elongated tube or cannula 120, a tissue penetrating stylet 110, and a fixed volume vacuum source 190. The control handle 130 may include a body 138 having two finger holes 131 disposed on opposite sides thereof. An attachment member 133 configured to releasably engage and attach the fixed volume vacuum source 190 to the control handle 130 may be disposed on the external surface of the control handle 130. The attachment member 133 defines an external end of a vacuum port 150 shown in FIGS. 7(a)-9(c).

Returning to FIGS. 1 and 2, the control handle 130 also includes an actuating member 132 that is in mechanical communication with one or more actuation mechanism(s) that permit relative movement between the cannula 120 and the stylet 110. The actuation mechanism(s) for the cannula 120 and the stylet 110 may be a spring-loaded mechanism, such as, for example and without limitation, the mechanism described in U.S. Pat. No. 5,538,010, the entirety of which is hereby incorporated by reference. As described in detail below with regard to FIGS. 7-10, the control handle 130 also includes an actuation/timing mechanism for releasing the fixed volume vacuum source 190. The actuation mechanism for the stylet 110, cannula 120, and the vacuum source 190 may be integrated into a single mechanism, or may be separated into one or more distinct mechanisms. The actuation mechanism(s) may be housed within the body 138 of the control handle 130. The control handle 130 may be formed of a sterilizable polymer, for example and without limitation, Nylon, polypropylene, and acrylonitrile butadiene styrene (ABS).

As shown in FIG. 1, the fixed volume vacuum source 190 may be an inexpensive, disposable, and readily available vacuum source, such as a glass stored vacuum vial. Alternatively, as shown in FIG. 2, the fixed volume vacuum source may be a commonly available disposable syringe.

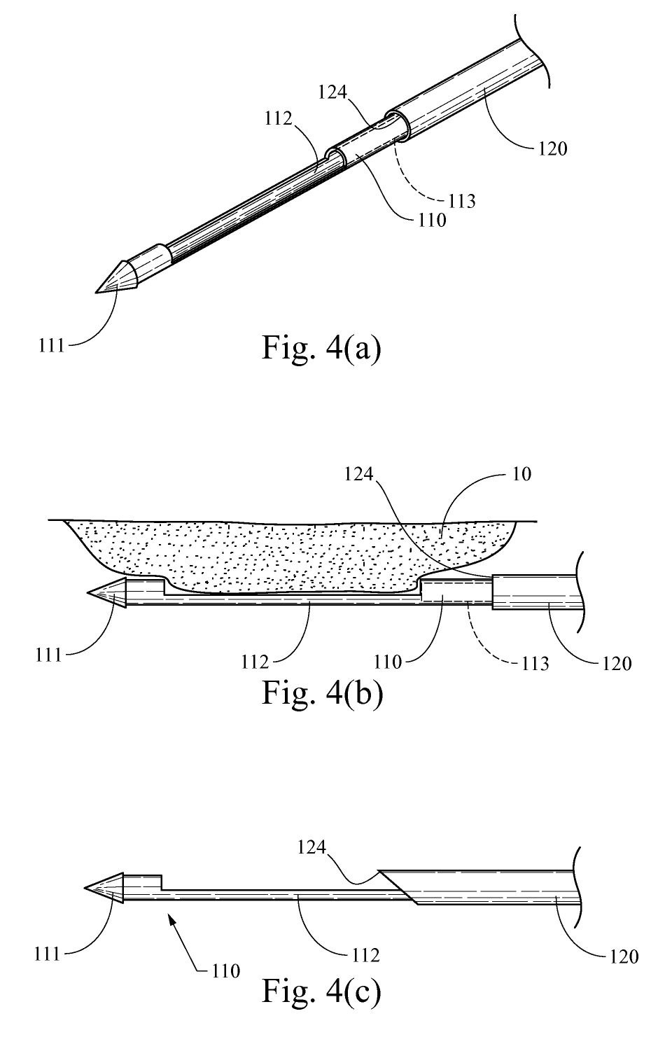

Turning to FIG. 4, the stylet 118 and the cannula 120 are slidably received into the distal end of the control handle 130. The cannula 120 includes a distal portion including a distal end 124 and a proximal end. The cannula 120 extends proximally into the control handle 130 and is formed from a bio-compatible material, for example and without limitation, metal alloys such as stainless steel, titanium, nickel-titanium alloys, and other suitable metals, or rigid/semi-rigid plastics. However, it should be understood that any other suitable existing or later developed materials may also be used.

The cannula 120 may have a diameter of between 14-20 gauge, and may be 18 gauge. In some embodiments, the cannula 120 may have substantially the same diameter along its length, and in other embodiments, the cannula 120 may taper from a larger gauge/diameter at the proximal end to a smaller diameter at the distal end 124 to provide added stability. In a one embodiment, the cannula 120 may be a stainless steel tube having a diameter of about 0.330 inches and a wall thickness of about 0.010 inches. At least the distal portion of the cannula 120 may be sized slightly larger than the stylet 110 to minimize the annular gap between the stylet 110 and the cannula 120, and to stabilize the stylet 110 while still allowing the stylet 110 to slidably move between the retracted and deployed positions within a lumen defined by the cannula 120. The cannula 120 may be 0.005-0.01 inches larger than the outer diameter of the stylet 110, and in one embodiment may be between 0.003 and 0.004 inches larger. Both the cannula 120 and the stylet 110 may be electro polished or otherwise treated to minimize friction therebetween, and to eliminate any burrs produced during the manufacturing process. The distal end 124 of the cannula 120 is shaped to cut or shear tissue 10 in a longitudinal direction as the cannula 110 is advanced through the portion of the tissue mass 10 disposed outside the sample containing region 112. As shown in FIG. 4(c), the distal end 124 of the cannula 120 may be formed as a distally pointed shearing edge having a beveled annular shape. An inner surface of the beveled edge may extend axially beyond an outer surface thereof to preclude the prolapsed tissue from catching on the cannula 120 as the cannula 120 and the stylet 110 are advanced to the target site.

The stylet 110 may be slidably attached to the control handle 130 and disposed within a central lumen of the cannula 120. The stylet 110 may include a sharp distal end 111 that is adapted to introduce the surgical cutting device 110 to a target site containing the tissue mass 10 to be biopsied by piercing through a patient's skin/tissue and advancing the stylet 110 and the cannula 120 into the tissue mass 10. The sharp distal end 111 may be a conical point, a bevel, a multi faced cutting surface or the like. As shown in FIG. 4, the stylet 110 may be formed of a generally cylindrical tube defining a central lumen 113 that extends along a central axis of the stylet 110 from at least a distal end of a sample containing region 112, to a proximal end of the stylet 110. In an embodiment, the stylet 110 may be formed from stainless steel having a diameter of about 0.300 inches.

As shown in the embodiment of FIG. 4, the sample collection region 112 is disposed in a distal portion of the stylet 110 and may be formed by cutting away a portion of the generally cylindrical stylet 110. The sample collecting region 112 is sized to collect a sample volume of 2-5 mm³, and may have a longitudinal length of 0.5 to 0.75 inches. Like the cannula 120, the stylet 110 may be made from a biocompatible metal, for example and without limitation, stainless steel, titanium, and nickel-titanium alloys, or other suitable materials as known in the art. It should be understood that while the stylet 110 is illustrated as having a single sample containing region 112, it is not limited thereto, and may include a plurality of sample containing regions 112 for use in large tissue masses 10.

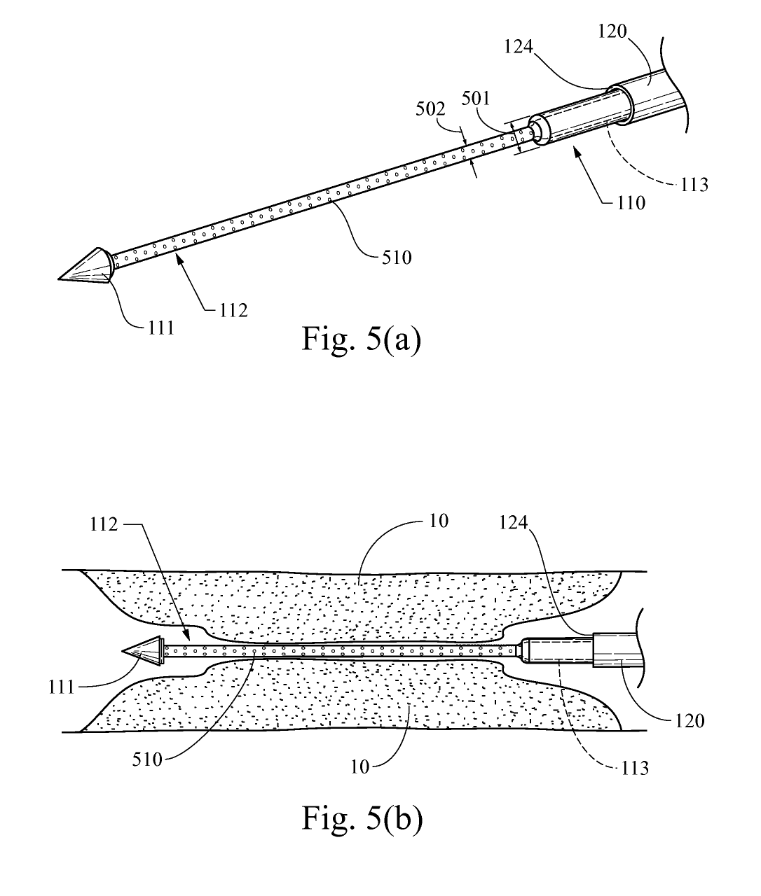

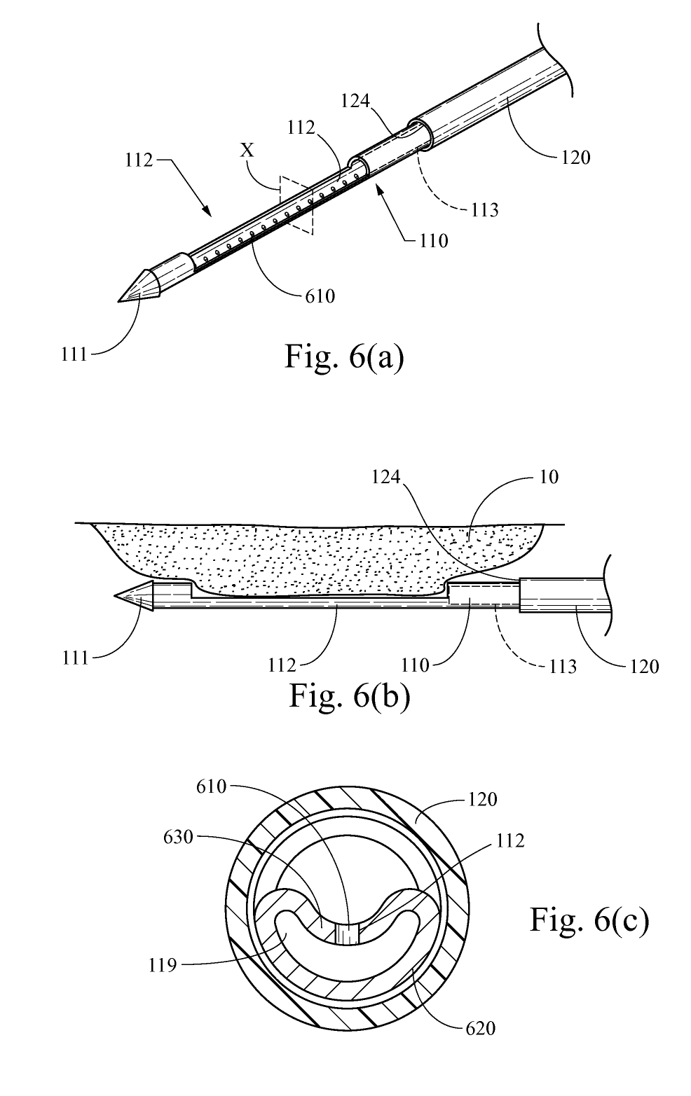

FIGS. 5 and 6 illustrate alternative embodiments of the stylet 110 depicted in FIG. 4 that are adapted to maximize the negative pressure affect of the fixed volume vacuum source 190 in drawing in and holding the prolapsed tissue in the sample containing region 112 prior to and/or during the cutting operation. As shown in FIG. 5, the stylet 110 may be formed from a hollow tube that transitions from a larger cross-sectional area to smaller cross-sectional area. For example, the hollow tube may transition from a larger diameter 501 in the proximal portion to a smaller diameter 502 in the sample containing region 112, thereby creating an annular space between the external surface of the reduced diameter sample containing region 112, and the inner surface of the cannula 120 for tissue collection. The sample containing region 112 may be formed integrally with the proximal portion by swaging or the like, as is known in the art. The smaller diameter sample containing region may also be formed from separate pieces that are welded or otherwise fixedly attached to each other at the distal end of the proximal portion of the stylet 110. In one embodiment, the junction between the proximal portion and the sample containing region 112 is completely sealed, thereby creating a single continuous lumen 113 that extends through the proximal portion of the stylet 110 and the sample collection region 112.

As shown in FIG. 5, the sample containing region 112 may include a plurality of apertures 510 extending through a wall of the sample collection region 112 such that the lumen 113 in the sample containing region 112 is in fluid communication with the annular space outside the sample containing region 112. The apertures 510 are spaced apart from each other and disposed along the length and around the circumference of the sample containing region 112. In operation, when the fixed volume vacuum source 190 is put in fluid communication with the lumen 113, fluid is drawn through the apertures 510 and along the lumen 113 in the proximal direction, thereby creating suction against the tissue surrounding the sample collection region 112. This suction tends to draw the tissue 10 into the sample containing region 112, thereby maximizing the amount of prolapsed tissue disposed in the sample collection region 112 and holding the prolapsed tissue in place as the cannula 120 is fired.

FIGS. 6(a)-(c) illustrate another embodiment of the stylet 110. As shown in FIGS. 6(a) and (c), the sample containing region 112 of the stylet 110 includes a crescent shaped lumen 119 defined by an inner surface 630 and an outer surface 620. The crescent shaped lumen 119 may be formed by deforming a portion of an outer surface of the stylet 110 corresponding to the sample containing region 112 by compressing it in a radially inward direction. A plurality of apertures 610 extend through a wall of the inner surface 630 such that the lumen 119 in the sample containing region 112 is in fluid communication with the space that is external of the inner surface 630 of the sample containing region 112. The proximal end of the sample containing region 112 may terminate in an aperture disposed at the distal end of the lumen 113, or may be swaged, or otherwise drawn down in a continuous manner to create a seal between the lumen 113 of the proximal portion and the lumen 119 of the sample containing region 112. In operation, when the fixed volume vacuum source 190 is put in fluid communication with the lumen 113, fluid is drawn through the apertures 610 and along the lumen 113 in the proximal direction, thereby creating suction against the tissue surrounding the sample collection region 112. As described above in connection with FIGS. 5(a) and (b), this suction tends to draw the tissue 10 into the sample containing region 112, thereby maximizing the amount of prolapsed tissue disposed in the sample collection region 112 and holding the prolapsed tissue in place as the cannula 120 is fired.

FIG. 3 illustrates an embodiment having an integrally formed fixed volume vacuum source 190. Like the embodiments of FIGS. 1 and 2, the embodiment of FIG. 3 includes a control handle 130 connected to an elongated tube or cannula 120, a tissue penetrating stylet 110, and a fixed volume vacuum source 190. However, unlike the embodiments of FIGS. 1 and 2, in this embodiment, the fixed volume vacuum source 190 is integrally formed in the control handle 130. The fixed volume vacuum source 190 is in direct connection with the vacuum port 150 shown in FIGS. 7(a)-9(c). In one embodiment, the integrally formed fixed volume vacuum source 190 is a sealed vacuum chamber housed within the control handle 130. In other embodiments, the fixed volume vacuum source 190 may be a piston/plunger system in which negative pressure is created by movement of the actuating member 132 relative to the control handle 130.

Note that because the above described embodiments of FIG. 3 incorporate the fixed volume vacuum source 190 into the control handle 130, the control handle may be more complex to manufacture as compared with the embodiments of FIGS. 1 and 2. It should be understood that while the sealed vacuum chamber embodiment of FIG. 3 has a fixed maximum volume for each individual firing of the stylet 110, the vacuum chamber may be “recharged” by withdrawing the piston/plunger again for subsequent firings during the same or a different procedure.

In contrast, the embodiments of FIGS. 1 and 2 may offer advantages in manufacturing cost and use. For example, because the embodiments of FIGS. 1 and 2 utilize disposable glass vacuum vials or syringes, which are inexpensive and commonly available to physicians, the fixed volume vacuum sources 190 can be easily exchanged in the event the vacuum source 190 is defective or inadvertently damaged during shipping or use. Moreover, the vacuum source 190 can be replaced with a new sterile vacuum source 190 for multiple firings during a biopsy procedure, thus minimizing the risk of infection or contamination at little additional cost. Additionally, the replaceable and releasably attached fixed volume vacuum sources 190 of FIGS. 1 and 2 may offer cost and time benefits in manufacturing as the control handles 130 do not incorporate integral vacuum sources 190 and therefore require fewer interactive components.

FIGS. 7(a)-10(b) illustrate a plurality of actuation mechanisms of the control handle 130 that may be used with any of the embodiments described above.

FIGS. 7(a)-(d) illustrate an embodiment of the surgical cutting device 700 having a user determinable duration for vacuum source application. As shown in FIG. 7(a), the proximal end of the stylet 110 is attached to the actuator member 132, which seals the proximal end of the lumen 113. The stylet 110 also includes a vacuum activation marker 115 and a vacuum port 140 in the form of an aperture disposed in a wall of the stylet 110. The control handle 130 includes a vacuum chamber 135 connected to and in fluid communication with a handle vacuum port 150. At least one seal 160 is disposed at the proximal and distal ends of the vacuum chamber 135, with each seal 160 being sized to fixedly engage an inner wall of the vacuum chamber 135, and slidingly engage the external surface of the stylet 110. In this embodiment, the stylet 110 may extend through an entire length of the control handle 130, including the vacuum chamber 135, an annular space within the spring 170, and the cannula 120.

Initially, as shown in FIG. 7(a), the cannula 120 is in a cocked position in which a base 122 of the cannula 120 is biased against the compressed spring 170, and the stylet 110 is in a retracted position in which a distal end of the sample collection region 112 is disposed proximal of the distal end 124 of the cannula 120, such that the entire sample collection region 112 is disposed within the cannula 120. When the stylet 110 is moved from the retracted position to the deployed position, the stylet 110 is advanced distally with respect to the cannula 120 in the cocked position and the control handle 130. As shown in FIG. 7(b), the sharp distal tip 111 of the stylet 110 is advanced out of the cannula 120 and into the tissue mass 10, thereby exposing the tissue collection region 112 and moving the stylet vacuum port 140 toward the vacuum chamber 135. As the stylet 110 is advanced to the point that the vacuum activation marker 115 is no longer visible from the outside of the control handle 130, the stylet vacuum port 140 is advanced beyond the proximal seal 160 in the vacuum chamber 135, thereby exposing the lumen 113 to the fixed volume vacuum source 190 through the handle vacuum port 150, the vacuum chamber 135, and the stylet vacuum port 140. Once fluid communication has been established from the lumen 113 to the fixed volume vacuum source 190, fluid begins to flow proximally through the lumen toward the vacuum source 190, thereby creating suction and drawing the tissue 10 into the tissue collection region 112. Once the vacuum has been activated, the tissue 10 is drawn into the tissue collection region 112 until the tissue 10 has plugged or otherwise sealed off the lumen 113 and/or the apertures 510/610. The physician may then fire the cannula 120 by continuing to advance the actuation member 132 in the distal direction, or through a separate triggering mechanism. Upon firing, the spring 170 is released from its compressed configuration and forces the cannula 120 in the distal direction, thereby advancing the sharp distal end 124 of the cannula 120 and shearing off a sample of tissue disposed in the tissue collection region 112 from the tissue mass 10. The cannula 120 may be advanced from the cocked position to the cutting position by the spring 170 in less than 0.5 seconds. Note that because the fixed volume vacuum source 190 is activated by relative movement of the stylet 110, which is wholly independent of the firing of the cannula 120, the physician is able to selectively determine the time for vacuum application before shearing off the tissue sample.

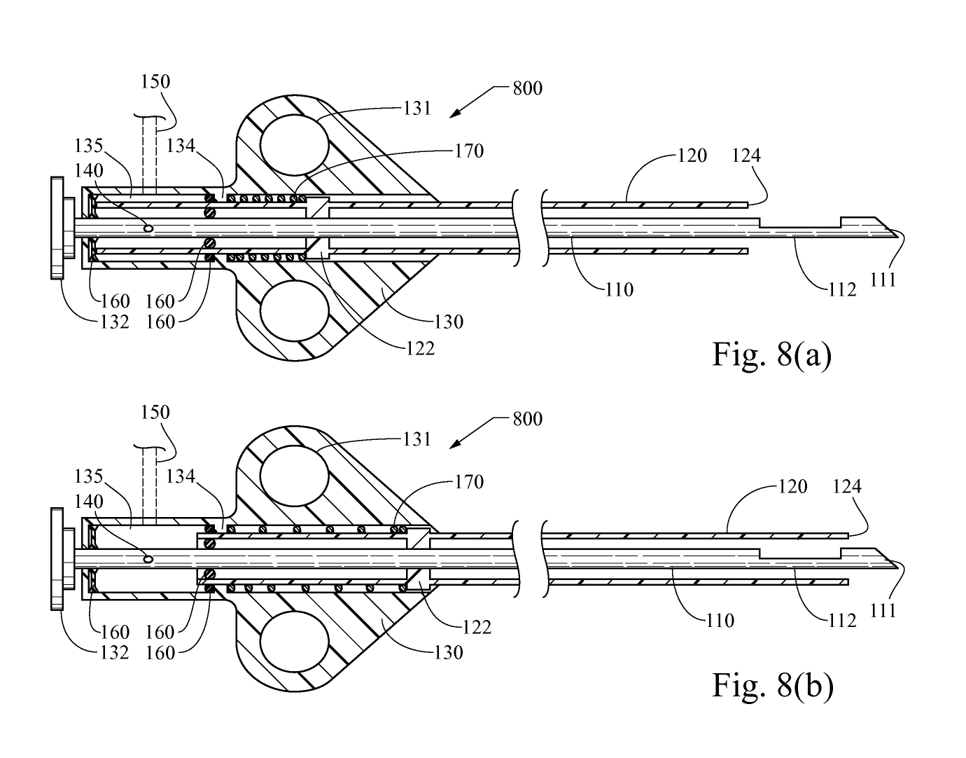

FIGS. 8(a)-(b) illustrate an embodiment of the surgical cutting device 800 having an integrated vacuum actuation and firing mechanism. As shown in FIG. 8(a), the proximal end of the stylet 110 is attached to the actuator member 132, which seals the proximal end of the lumen 113. A vacuum port 140 in the form of an aperture is disposed in a wall of the stylet 110. The control handle 130 includes a vacuum chamber 135 connected to and in fluid communication with the handle vacuum port 150. At least one seal 160 is disposed at the proximal and distal ends of the vacuum chamber 135, with each seal 160 being sized to sealingly engage an inner wall of the vacuum chamber 135 in a fixed manner, and sealingly engage the external surface of the stylet 110 and the internal and external surfaces of the cannula 120 in a slidable manner. The stylet 110 extends through an entire length of the control handle 130. That is the stylet extends through the vacuum chamber 135, an annular space within the spring 170 and the cannula 120. The cannula 120 is attached to a base 122 at an intermediate position such that when the cannula 120 is in the cocked position, the base 122 compresses the spring 170 in the proximal direction against a reaction surface 134 of the control handle 130.

Initially, the cannula 120 is in a cocked position in which a base 122 of the cannula 120 is biased against the compressed spring 170, and the stylet 110 is in a retracted position in which a distal end of the sample collection region 112 is disposed proximal of the distal end 124 of the cannula 120, such that the entire sample collection region 112 is disposed within the cannula 120. As shown in FIG. 8(a), when the stylet 110 is moved from the retracted position to the deployed position, the stylet 110 is advanced distally with respect to the cannula 120 in the cocked position such that the sharp distal tip 111 of the stylet is advanced into the tissue mass 10 and the tissue collection region 112 is exposed. As the stylet 110 is moved from the retracted to the deployed position, the stylet vacuum port 140 is advanced beyond the proximal seal 160 in the vacuum chamber 135. However, because the cannula 120 is still in the cocked position in which the proximal end of the cannula 120 and a portion of the cannula 120 adjacent to the distal end of the vacuum chamber 135 are still in contact with the seals 160, the stylet vacuum port 140 remains sealed from the fixed volume vacuum source 190.

Once the stylet 110 is completely deployed, the cannula 120 may be fired by continuing to advance the actuation member 132 in the distal direction. Upon firing, the spring 170 is released from its compressed configuration and forces the cannula 120 in the distal direction, thereby causing the proximal end of the cannula 120 to disengage from the proximal seal 160 and exposing the lumen 113 to the fixed volume vacuum source 190 through the handle vacuum port 150, the vacuum chamber 135, and the stylet vacuum port 140. Once fluid communication has been established from the lumen 113 to the fixed volume vacuum source 190, fluid begins to flow proximally through the lumen 113 toward the vacuum source 190. This creates suction and draws the tissue 10 into the tissue collection region 112 through the lumen 113 and/or apertures 510/610. The tissue 10 continues to be drawn into the tissue collection region 112 and is held in place by the suction as the cannula 120 is advanced in the distal direction and shears off a sample of tissue disposed in the tissue collection region 112 from the tissue mass 10. Because the vacuum activation and cannula firing are achieved substantially simultaneously through triggering of the same mechanism, the suction and shearing of the tissue sample occur within the firing time of the cannula, which may be less than 0.5 seconds.

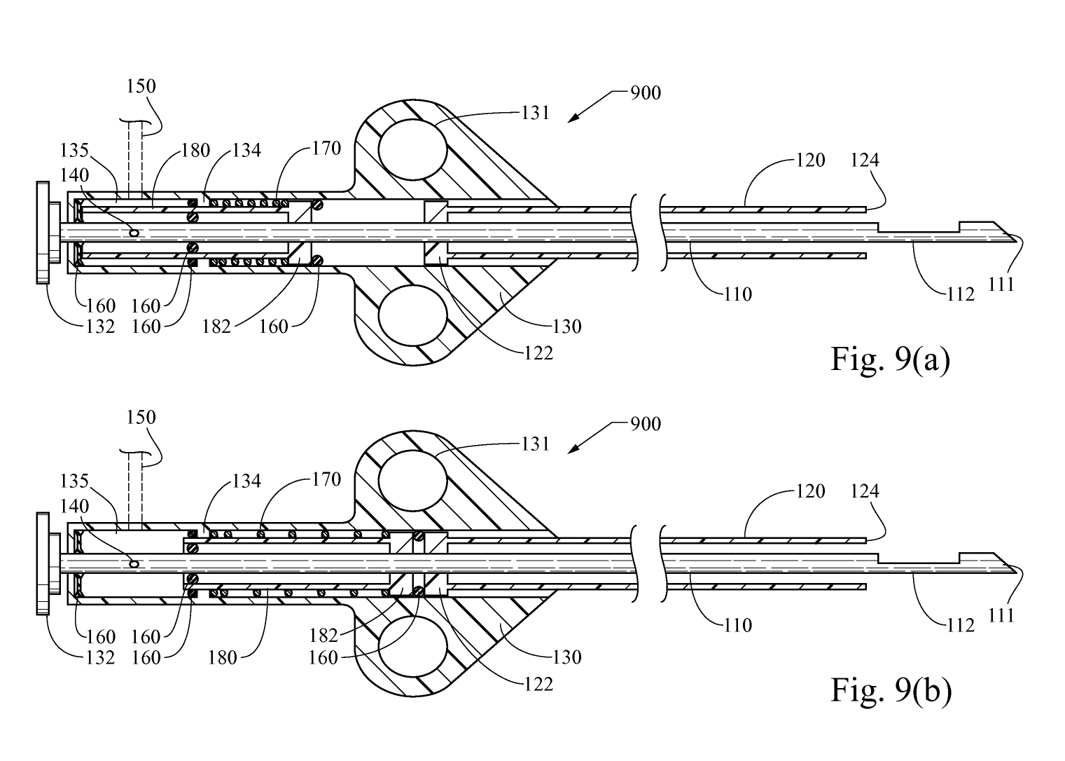

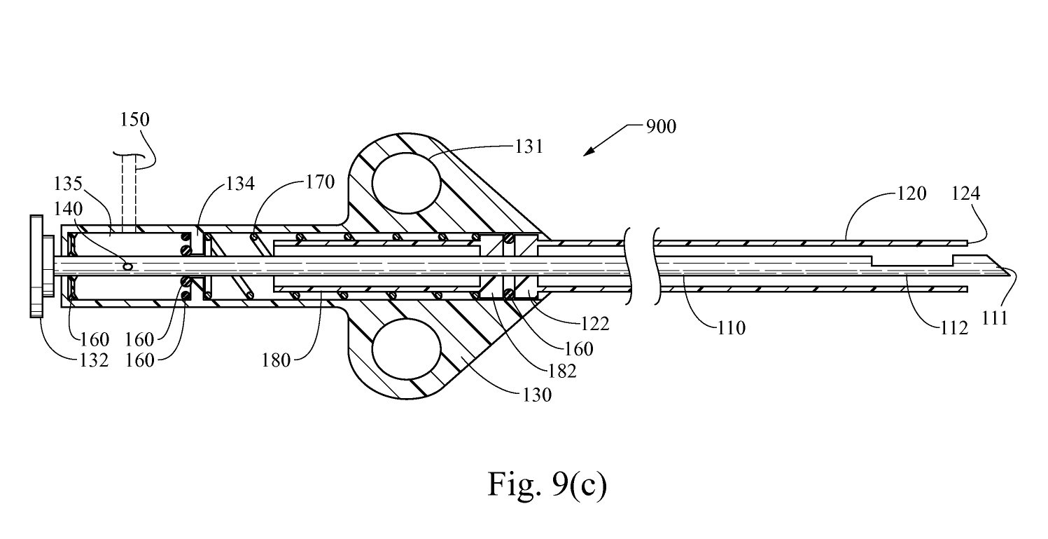

FIGS. 9(a)-(c) illustrate an embodiment of the surgical cutting device 900 having a delayed firing mechanism. As shown in FIG. 9(a), the proximal end of the stylet 110 is attached to the actuator member 132, which seals the proximal end of the lumen 113. A vacuum port 140 in the form of an aperture is disposed in a wall of the stylet 110. The control handle 130 includes a vacuum chamber 135 connected to and in fluid communication with the handle vacuum port 150. At least one seal 160 is disposed at the proximal and distal ends of the vacuum chamber 135, with each seal 160 being sized to sealingly engage an inner wall of the vacuum chamber 135 in a fixed manner, and sealingly engage the external surface of the stylet 110 and the internal and external surfaces of a sealing sleeve 180 in a slidable manner. The sealing sleeve 180 is attached to a slider 182 at its distal end, which is spaced longitudinally away from the base 122 attached to the proximal end of the cannula 120. A third seal 160 is disposed adjacent to a distal end of the slider 182. The sealing sleeve 180 may be made from a metallic cannula or a rigid polymer having sufficient column strength to both resist buckling and effectively transfer the stored energy of the spring 170 to the cannula 120 through the slider 182 and the base 122. When the sealing sleeve 180 and the cannula 120 are in the cocked position, the slider 182 compresses a spring 170 in the proximal direction against a reaction surface 134 of the control handle 130.

The stylet 110 may extend through an entire length of the control handle 130. That is the stylet 110 may extend through the vacuum chamber 135, an annular space within the spring 170, the sealing sleeve 180, and the cannula 120.

Initially, the cannula 120 is in a cocked position in which a base 122 of the cannula 120 is biased against the compressed spring 170, and the stylet 110 is in a retracted position in which a distal end of the sample collection region 112 is disposed proximal of the distal end 124 of the cannula 120 such that the entire sample collection region 112 is disposed within the cannula 120. As shown in FIG. 9(a), when the stylet 110 is moved from the retracted position to the deployed position, the stylet 110 is advanced distally with respect to the cannula 120 and sealing sleeve 180 in the cocked position such that the sharp distal tip 111 of the stylet 110 is advanced into the tissue mass 10, thereby exposing the tissue collection region 112.

As the stylet 110 is moved from the retracted to the deployed position, the stylet vacuum port 140 is advanced beyond the proximal seal 160 in the vacuum chamber 135. However, because the sealing sleeve 180 is still in the cocked position in which the proximal end and an intermediate portion of the sealing sleeve 180 are still in contact with the seals 160, the stylet vacuum port 140 remains sealed from the fixed volume vacuum source 190.

Once the stylet 110 is completely deployed, the sealing sleeve 180 and the cannula 120 may be fired by continuing to advance the actuation member 132 in the distal direction. Upon firing, the spring 170 is released from its compressed configuration and forces the sealing sleeve 180 in the distal direction, thereby causing the proximal end of the sealing sleeve 180 to disengage from the proximal seal 160 of the vacuum chamber 135, and exposing the lumen 113 to the fixed volume vacuum source 190 through the handle vacuum port 150, the vacuum chamber 135, and the stylet vacuum port 140.

Once fluid communication has been established from the lumen 113 to the fixed volume vacuum source 190, fluid begins to flow proximally through the lumen toward the vacuum source 190, which creates suction and draws the tissue 10 into the tissue collection region 112 through the lumen 113 and/or apertures 510/610. The tissue 10 continues to be drawn into the tissue collection region 112 and is held in place by suction as the sealing sleeve 180 is advanced in the distal direction. Once the sealing sleeve 180 and the seal 160 have been advanced through the longitudinal space separating the slider 182 and the base 122, the seal 160 is forced against a proximal surface of the base 122 and the energy of the spring 170 is transferred to the cannula 120, thereby forcing the cannula 120 to advance in the distal direction. As the cannula 120 and the sealing sleeve 180 move in the distal direction, the distal end of the cannula 124 contacts and shears off a sample of tissue disposed in the tissue collection region 112 from the tissue mass 10. While the vacuum activation and cannula firing are achieved through the same mechanism in this embodiment, the shearing of the tissue sample is delayed an additional amount from the activation of the suction as compared to the embodiment 800 of FIGS. 8(a) and (b) due to the longitudinal displacement between slider 182 of the sealing sleeve 180 and the base 122 of the cannula 120. The sealing sleeve 180 and the cannula 120 may be spaced apart so as to produce a delay of between about 50 to 2000 milliseconds. In one embodiment, the delay may be between 50 and 500 milliseconds. This difference in time delay may vary depending on the geometry (e.g. length, diameter, orientation of the ports, etc.) or tissue consistency. Moreover, the timing may be tailored such that it is optimized for particular biopsy procedures.

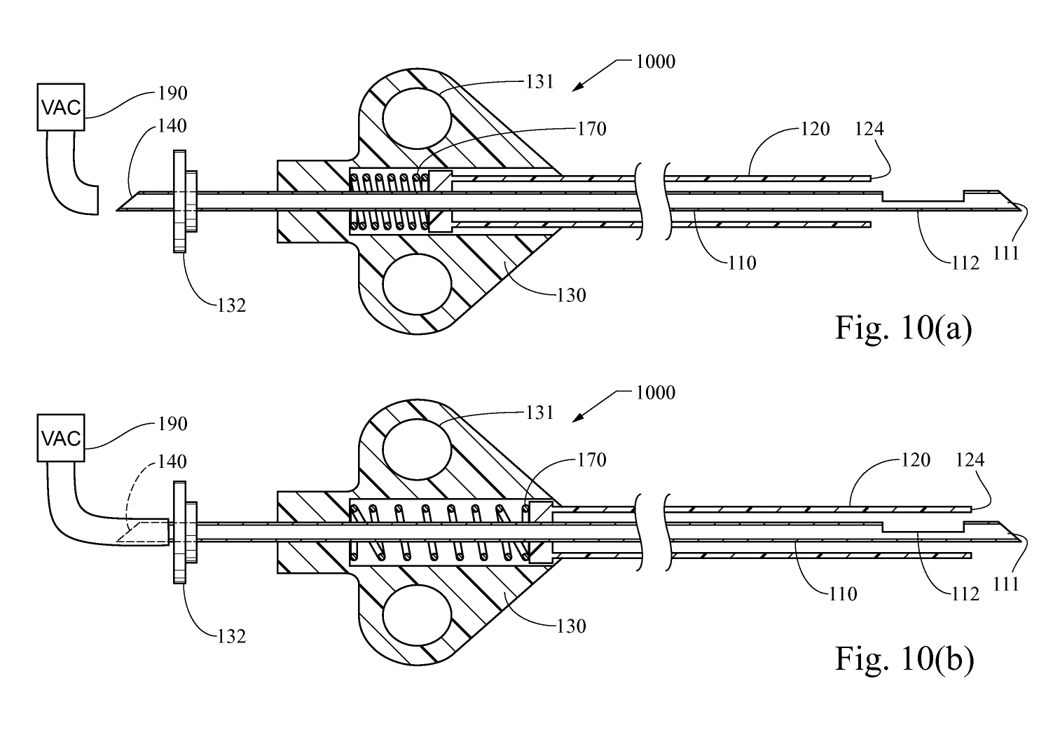

FIGS. 10(a) and (b) illustrate another embodiment of the surgical cutting device of FIGS. 1-3. As shown in FIGS. 10(a) and (b), the proximal end of the stylet 110 extends through an actuator member 132. The lumen 113 of the stylet 110 extends to the proximal end of the stylet 110 such that the proximal end of the stylet 110 forms a vacuum port 140. The proximal end of the stylet 110 has a sharp profile, for example and without limitation, an annular bevel, that is capable of piercing a flexible polymer or rubber seal of a fixed volume vacuum source 190, such as a glass vial, thereby simultaneously attaching the fixed volume vacuum source 190 to the stylet 110 and placing the lumen 113 in direct fluid communication with the vacuum source 190. The stylet 110 may extend through an entire length of the control handle 130, including an annular space within a spring 170 and the cannula 120.

Initially, the cannula 120 is in a cocked position in which a base 122 of the cannula 120 is based against the compressed spring 170, and the stylet 110 is in a retracted position in which a distal end of the sample collection region 112 is disposed proximal of the distal end 124 of the cannula 120, such that the entire sample collection region 112 is disposed within the cannula 120. As shown in FIG. 10(a), when the stylet 110 is moved from the retracted position to the deployed position, the stylet 110 is advanced distally with respect to the cannula 120 in the cocked position such that the sharp distal tip 111 of the stylet 110 is advanced into the tissue mass 10, thereby exposing the tissue collection region 112.

Once the stylet 110 is completely deployed, the proximal end of the stylet 110 pierces the fixed volume vacuum source 190, thereby exposing the lumen 113 to the fixed volume vacuum source 190 through the vacuum port 140. After fluid communication has been established from the lumen 113 to the fixed volume vacuum source 190, fluid begins to flow proximally through the lumen toward the vacuum source 190, which creates suction and draws the tissue 10 into the tissue collection region 112 through the lumen 113 and/or apertures 510/610. In embodiments where the resistance force required to activate the cannula firing mechanism is set below a threshold level substantially equal to the force necessary to cause the proximal end of the stylet 110 to pierce the vacuum source 190, the piercing of the vacuum source 190 and the firing of the cannula 120 occur simultaneously. However, in embodiments where the resistance force required to activate the cannula firing mechanism is set above the threshold level, initially only the suction is activated and additional force input is required to fire the cannula 120. Once the triggering mechanism has been fired, the cannula 120 advances to its cutting position and shears off a sample of tissue disposed in the tissue collection region 112 from the tissue mass 10.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the features described above are not necessarily the only features of the invention, and it is not necessarily expected that all of the described features will be achieved with every embodiment of the invention.

1. A surgical cutting instrument, comprising:
a cannula having a distal end shaped to cut tissue, said cannula being attached to an actuation mechanism that moves said cannula from a cocked position to a cutting position;
a stylet comprising a distal portion having a sharp distal end, a sample collection region spaced proximally away from said sharp distal end, a first vacuum port, and a lumen extending from said first vacuum port to said sample collection region such that said sample collection region is in fluid communication with said first vacuum port, said stylet being disposed within said cannula, wherein said stylet is movable between a retracted position in which an entirety of said sample collection region is disposed within said cannula in said cocked position and a deployed position in which at least a portion of said sample collection region is disposed distal of said distal end of said cannula in said cocked position;
a releasably attachable fixed volume vacuum source;
a control handle comprising a second vacuum port, said fixed volume vacuum source being releasably attached to said control handle such that said second vacuum port is in fluid communication with said fixed volume vacuum source,
wherein when said stylet is in said retracted position said first and second vacuum ports are not in fluid communication with each other thereby sealing said fixed volume vacuum source from said lumen of said stylet, and when said stylet is in said deployed position said first and second vacuum ports are in fluid communication thereby allowing fluid flow from said sample collection region of said stylet to said fixed volume vacuum source through said lumen.

2. The surgical cutting device of claim 1, wherein said movement of said stylet from said retracted position to said deployed position causes said first and second vacuum ports to come into in fluid communication with each other.

3. The surgical cutting device of claim 1, wherein said fixed volume vacuum source is a disposable sealed vacuum vial.

4. The surgical cutting device of claim 1, wherein said fixed volume vacuum source is a disposable syringe.

5. The surgical cutting device of claim 1, wherein said proximal portion of said stylet has a first cross sectional area and said sample containing region has a second cross sectional area, said second cross sectional area being smaller than said first, wherein said sample containing region comprises a plurality of apertures disposed on an outer surface thereof, said plurality of apertures being in fluid communication with said lumen.

6. The surgical cutting device of claim 1, wherein said sample containing region has a first surface and a second surface, wherein said first and second surfaces are displaced from each other to form a lumen therebetween, said first surface being substantially contiguous with an outer surface of said stylet, said second surface being disposed radially inward of said outer surface of said stylet thereby creating a space to receive a sample, and wherein said second surface comprises a plurality of apertures along a length thereof, said apertures being in fluid communication with said lumen.

7. A surgical cutting instrument, comprising:
a cannula having a distal end shaped to cut tissue, said cannula being attached to an actuation mechanism that moves said cannula from a cocked position to a cutting position;
a stylet comprising a distal portion having a sharp distal end, a sample collection region, a first vacuum port, and a lumen extending from said first vacuum port to said sample collection region such that said sample collection region is in fluid communication with said first vacuum port, said stylet being disposed within said cannula, wherein when said cannula is in said cocked position at least a portion of said sample collection region is disposed distal of said distal end of said cannula, and wherein said first vacuum port is disposed distal of a proximal end of said cannula when said stylet is in said deployed position;
a control handle having a second vacuum port; and
a fixed volume vacuum source releasably attached to said control handle and in fluid communication with said second vacuum port;
wherein, when said cannula is in said cocked position said second vacuum port is sealed from said first vacuum port, thereby preventing fluid communication between said fixed volume vacuum source and said lumen of said stylet, and when said cannula is in said cutting position said first and second vacuum ports become at least partially unsealed from one another thereby permitting fluid flow from said sample collection region of said stylet to said fixed volume vacuum source through said lumen and said first and second vacuum ports.

8. The surgical cutting device of claim 9, wherein said first vacuum port is disposed distal of a proximal end of said cannula when said stylet is in said deployed position,
wherein said control handle further comprises first and second seals, said first seal being disposed proximal of said second vacuum port and said second seal being disposed distal of said second vacuum port, wherein, when said cannula is in said cocked position and said stylet is in said deployed position said first and second seals sealingly engage a proximal portion of said cannula and sealingly engage a portion of said stylet comprising said first vacuum port, and
wherein when said cannula is in said cocked position said first vacuum port is sealed from said second vacuum port by said proximal portion of said cannula thereby preventing fluid communication between said fixed volume vacuum source and said lumen of said stylet, and when said cannula is in said cutting position said proximal portion of said cannula disengages from at least one of said first and second seals thereby permitting fluid flow from said sample collection region of said stylet to said fixed volume vacuum source through said lumen and said first and second vacuum ports.

9. The surgical cutting device of claim 7, wherein said movement of said cannula from said cocked to said cutting position causes said first and second vacuum ports to come into in fluid communication with each other.

10. The surgical cutting device of claim 7, wherein said fixed volume vacuum source is selected from one of a group consisting of a disposable sealed vacuum vial and a disposable syringe.

11. The surgical cutting device of claim 7, wherein said proximal portion of said stylet has a first cross sectional area and said sample containing region has a second cross sectional area, said second cross sectional area being smaller than said first, wherein said sample containing region comprises a plurality of apertures disposed on an outer surface thereof, said plurality of apertures being in fluid communication with said lumen.

12. The surgical cutting device of claim 7, wherein said sample containing region has a first surface and a second surface, wherein said first and second surfaces are displaced from each other to form a lumen therebetween, said first surface being substantially contiguous with an outer surface of said stylet, said second surface being disposed radially inward of said outer surface of said stylet thereby creating a space to receive a sample, and wherein said second surface comprises a plurality of apertures along a length thereof, said apertures being in fluid communication with said lumen.

13. The surgical cutting instrument of claim 7, further comprising:
a sealing sleeve attached to a second actuation mechanism that moves said sealing sleeve from a sealed position to a released position,
wherein, when said sealing sleeve is in said sealed position said first vacuum port is sealed from said second vacuum port by said sealing sleeve, said sealing sleeve thereby preventing fluid communication between said fixed volume vacuum source and said lumen of said stylet, and when said sealing sleeve is in said released position said first and second vacuum ports become at least partially unsealed from one another thereby permitting fluid flow from said sample collection region of said stylet to said fixed volume vacuum source through said lumen and said first and second vacuum ports.

14. The surgical cutting device of claim 13, wherein said first vacuum port is disposed within said sealing sleeve when said when said cannula is in said cocked position,
wherein said control handle further comprises first and second seals, said first seal being disposed proximal of said second vacuum port and said second seal being disposed distal of said second vacuum port, respectively, wherein, when said sealing sleeve is in said sealed position, said first and second seals sealingly engage said sealing sleeve and sealingly engage a portion of said stylet comprising said first vacuum port, and
wherein when said sealing sleeve is in said released position, said sealing sleeve disengages from at least one of said first and second seals thereby permitting fluid flow from said sample collection region of said stylet to said fixed volume vacuum source through said lumen and said first and second vacuum ports.

15. The surgical cutting device of claim 13, wherein said first and second actuation mechanisms are a single mechanism.

16. The surgical cutting device of claim 13, wherein said fixed volume vacuum source is selected from a group consisting of a disposable sealed vacuum vial and a disposable syringe.

17. The surgical cutting device of claim 13, wherein said proximal portion of said stylet has a first cross sectional area and said sample containing region has a second cross sectional area, said second cross sectional area being smaller than said first, wherein said sample containing region comprises a plurality of apertures disposed on an outer surface thereof, said plurality of apertures being in fluid communication with said lumen.

18. The surgical cutting device of claim 13, wherein said sample containing region has a first surface and a second surface, wherein said first and second surfaces are displaced from each other to form a lumen therebetween, said first surface being substantially contiguous with an outer surface of said stylet, said second surface being disposed radially inward of said outer surface of said stylet thereby creating a space to receive a sample, and wherein said second surface comprises a plurality of apertures along a length thereof, said apertures being in fluid communication with said lumen.

19. A surgical cutting instrument, comprising:
a cannula having a distal end shaped to cut tissue, said cannula being attached to an actuation mechanism that moves said cannula from a cocked position to a cutting position;
a stylet comprising a distal portion having a sharp distal end, a sample collection region spaced proximally away from said sharp distal end, a vacuum port disposed at a proximal end of said stylet, and a lumen extending from said vacuum port to said sample collection region such that said sample collection region is in fluid communication with said vacuum port, said stylet being disposed within said cannula, and wherein said stylet is movable between a retracted position in which an entirety of said sample collection region is disposed within said cannula in said cocked position and a deployed position in which at least a portion of said sample collection region is disposed distal of said distal end of said cannula in said cocked position;
a releasably attachable fixed volume vacuum source,
wherein said vacuum port is not sealed from said lumen, and said proximal end of said stylet is shaped to attach directly to said fixed volume vacuum source, said direct attachment creating immediate fluid communication with said vacuum port, thereby allowing fluid flow from said sample collection region of said stylet to said fixed volume vacuum source through said lumen.

20. The surgical cutting instrument of claim 19, wherein said fixed volume vacuum source is a sealed vacuum vial and wherein said proximal end of said stylet has a sharp edge that pierces said sealed vacuum vial, thereby directly attaching said sealed vacuum vial to said stylet and creating fluid communication between said sealed vacuum vial and said vacuum port.

21. A stylet for a biopsy needle comprising:
a stylet having a proximal portion and a sample containing region,
wherein the proximal portion of the stylet has a first cross sectional area and said sample containing region has a second cross sectional area, said second cross sectional area being smaller than said first, wherein said sample containing region comprises a plurality of apertures disposed around an outer surface thereof, said plurality of apertures being in fluid communication with said lumen.

22. A stylet for a biopsy needle comprising:
a stylet having a proximal portion having a first cross sectional area and a sample containing region having a second cross sectional area, the second cross sectional area being less than the first cross sectional area,
wherein said sample containing region has a first surface and a second surface, wherein said first and second surfaces are displaced from each other to form a lumen therebetween, said first surface being substantially contiguous with an outer surface of said stylet, said second surface being disposed radially inward of said outer surface of said stylet thereby creating a space to receive a sample, and wherein said second surface comprises a plurality of apertures along a length thereof, said apertures being in fluid communication with said lumen.
